(19) United States
(12) Patent Application Publication
(10) Pub. No.: US 2026/0135005 A1
(43) Pub. Date: May 14, 2026

(54) ARTIFICIAL INTELLIGENCE MODELING TO ASSIST RADIOTHERAPY TREATMENT PLANNING

(71) Applicant: Siemens Healthineers International AG, Steinhausen (CH)

(72) Inventors: Esa Kuusela, Espoo (FI); Lauri Halko, Helsinki (FI); Ismo Hautala, Espoo (FI)

(73) Assignee: Siemens Healthineers International AG, Steinhausen (CH)

(21) Appl. No.: 18/943,765

(22) Filed: Nov. 11, 2024

(51) Int. Cl. G16H 80/00; A61N 5/10; G06F 40/35; G16H 20/40

(52) U.S. Cl. CPC G16H 80/00; A61N 5/1031; A61N 5/1039; G06F 40/35; G16H 20/40

(57) ABSTRACT

Embodiments described herein provide for implementing a machine-learning language processing model in a use-case for radiotherapy treatment planning (RTTP) discussion and treatment planning assistance. In an embodiment, a device presents a user interface for medical professionals to communicate during RTTP discussions; a server receives input from one professional, generates proficiency scores for both sender and receiver and uses the model to predict contextual suggestions based on these scores. The model, trained on previous RTTP discussions, provides communication clarity. The device displays the suggestion for approval and, upon receiving confirmation, transmits the treatment attribute to a plan optimizer to generate a radiotherapy treatment plan for the patient. This process enhances communication and improves the efficiency and accuracy of RTTP.

### TECHNICAL FIELD

This application relates generally to generating a radiotherapy treatment plan using a machine-learning language processing model.

### BACKGROUND

Radiotherapy or radiation therapy (RT) is one of the main modalities used in cancer treatment, and RT treatment planning (RTTP) is a complex process that contains specific guidelines, protocols, and instructions adopted by different medical professionals, such as clinicians, medical device manufacturers, and the like. The RTTP process typically necessitates collaboration among multiple specialists, including oncologists, physicists, dosimetrists, and therapists, along with various software components. Typically, the overall treatment strategy, such as selecting various treatment attributes, is discussed and decided in a multi-disciplinary RTTP discussion, where various medical professionals discuss how to treat the patient. These meetings should thus be as efficient as possible in order to improve the RTTP planning process.

### SUMMARY

One of the challenges in clinical settings, particularly in RTTP discussions, is the communication gap between various medical professionals, such as oncologists, physicists, and dosimetrists. These expert medical professionals often have different backgrounds and use varied terminologies, which can lead to misunderstandings and inefficiencies. For instance, an oncologist might give instructions that are too vague or overly detailed, causing confusion for the physicist or dosimetrist responsible for executing the plan. This miscommunication can result in incorrect treatment assumptions, necessitating additional rounds of clarification and adjustments, ultimately delaying patient care and reducing operational efficiency. In some embodiments, verbal instructions provided during the RTTP discussions can often be imprecise, leading to situations where the recipient might misinterpret the instructions.

There is a substantial amount of precisely defined terminology that long-time radiation therapy practitioners are familiar with. However, it takes significant practice to use these terms fluently, and their excessive use could sometimes slow down communication. Another layer of communication issues arises from the need to be concise or the desire for detailed descriptions. When two experts who have communicated for a long time interact, their communication can be very concise. However, if a new team member replaces one expert, the language suddenly needs to become much more descriptive to ensure clarity.

Existing and conventional computer-implemented solutions have not been successful in solving the technical challenges discussed herein. Technical solutions to this problem have been limited, primarily relying on the templatization of the RTTP discussions. This approach involves using structured forms instead of natural language, where the form's design ensures that requests are made unambiguously when filled out. However, for any non-trivial task, these forms often require numerous fields to provide the necessary flexibility for detailed instructions. Additionally, some forms include free-text cells, meaning that the issue of ambiguous communication is not entirely resolved in practice.

The use of Large Language Models (LLMs), such as off-the-shelf products, to provide a natural language interface to certain software components has not been efficient or fruitful. For instance, the use of generic LLMs can introduce similar issues in human-machine interactions that are typically associated with human-to-human communication. Off-the-shelf LLMs can indeed provide generic results, which is highly undesirable in this field.

The methods and systems discussed herein improve conventional methods and systems by leveraging specially trained LLMs to enhance the precision and clarity of communication among radiation therapy specialists during RTTP discussions. Unlike traditional methods that rely on structured forms or free-text fields, the use of specially trained LLMs discussed herein enables dynamic and context-aware interpretation and translation of instructions. This allows for more nuanced and accurate communication tailored to the recipients'specific expertise and experience levels. Consequently, the methods and systems discussed herein improve this technical field by reducing the likelihood of misinterpretation. Further improvements are provided by ensuring that all parties have a clear and consistent understanding of the instructions, ultimately improving the efficiency and effectiveness of the RTTP process. Additionally, by adapting the communication style to suit the individual needs of different professionals, the system fosters smoother interactions and minimizes the need for repeated clarifications, which are common in current systems.

In some aspects, the techniques described herein relate to a method including: presenting, by a processor, a user interface providing an interaction graphical component between a plurality of medical professionals communicating regarding an RTTP of a patient during an RTTP discussion; receiving, by the processor from the interaction graphical component from a first medical professional to be received by a second medical professional, an input including a treatment attribute corresponding to the radiation therapy treatment of the patient; generating, by the processor, a first radiotherapy proficiency score for the first medical professional and a second radiotherapy proficiency score for the second medical professional, the first score and the second score corresponding to a professional attribute of the first medical professional and the second medical professional; executing, by the processor, a machine-learning language processing model using the input to predict a contextual suggestion corresponding to the treatment attribute in accordance with the first radiotherapy proficiency score and the second radiotherapy proficiency, wherein the machine-learning language processing model is trained using a set of transcriptions of a set of RTTP discussions for a set of previously implemented radiation therapy treatments; presenting, by the processor on the interaction graphical component, the contextual suggestion corresponding to the treatment attribute; and in response to receiving an indication of approval from the first medical professional and the second medical professional, transmitting, by the processor, the treatment attribute to a radiation therapy plan optimizer.

In some aspects, the techniques described herein relate to a method, wherein the first radiotherapy proficiency score or the second radiotherapy proficiency score corresponds to a time period of medical experience of the first medical professional or the second medical professional.

In some aspects, the techniques described herein relate to a method, wherein the first radiotherapy proficiency score or the second radiotherapy proficiency score is generated using the machine-learning language processing model via analyzing a previous conversation associated with the first medical professional or the second medical professional.

In some aspects, the techniques described herein relate to a method, further including presenting, by the processor on the interaction graphical component, a hyperlink configured to direct the interaction graphical component to third-party data associated with the radiation therapy treatment.

In some aspects, the techniques described herein relate to a method, wherein the machine-learning language processing model is further trained using previously performed radiation therapy treatments.

In some aspects, the techniques described herein relate to a method, further including in response to the input satisfying a threshold, presenting, by the processor in the interaction graphical component, a warning message.

In some aspects, the techniques described herein relate to a method, wherein the first input is a medical image.

In some aspects, the techniques described herein relate to a system, including: a server including at least one processor and a non-transitory computer-readable medium containing instructions that, when executed by the at least one processor, causes the at least one processor to: present a user interface providing an interaction graphical component between a plurality of medical professionals communicating regarding an RTTP of a patient during an RTTP discussion; receive, from the interaction graphical component from a first medical professional to be received by a second medical professional, an input including a treatment attribute corresponding to the radiation therapy treatment of the patient; generate a first radiotherapy proficiency score for the first medical professional and a second radiotherapy proficiency score for the second medical professional, the first score and the second score corresponding to a professional attribute of the first medical professional and the second medical professional; execute a machine-learning language processing model using the input to predict a contextual suggestion corresponding to the treatment attribute in accordance with the first radiotherapy proficiency score and the second radiotherapy proficiency score, wherein the machine-learning language processing model is trained using a set of transcriptions of a set of RTTP discussions for a set of previously implemented radiation therapy treatments; present, on the interaction graphical component, the contextual suggestion corresponding to the treatment attribute; and in response to receiving an indication of approval from the first medical professional and the second medical professional, transmit the treatment attribute to a radiation therapy plan optimizer.

In some aspects, the techniques described herein relate to a system, wherein the first radiotherapy proficiency score or the second radiotherapy proficiency score corresponds to a time period of medical experience of the first medical professional or the second medical professional.

In some aspects, the techniques described herein relate to a system, wherein the first radiotherapy proficiency score or the second radiotherapy proficiency score is generated using the machine-learning language processing model via analyzing a previous conversation associated with the first medical professional or the second medical professional.

In some aspects, the techniques described herein relate to a system, wherein the instructions further cause the processor to present, on the interaction graphical component, a hyperlink configured to direct the interaction graphical component to third-party data associated with the radiation therapy treatment.

In some aspects, the techniques described herein relate to a system, wherein the machine-learning language processing model is further trained using previously performed radiation therapy treatments.

In some aspects, the techniques described herein relate to a system, wherein the instructions further cause the at least one processor to, in response to the input satisfying a threshold, present in the interaction graphical component, a warning message.

In some aspects, the techniques described herein relate to a method, wherein the first input is a medical image.

In some aspects, the techniques described herein relate to a system, including: a computer configured to display a user interface; and a server in communication with the computer and a machine-learning language processing model, the server configured to: present the user interface providing an interaction graphical component between a plurality of medical professionals communicating regarding an RTTP of a patient during an RTTP discussion; receive, from the interaction graphical component from a first medical professional to be received by a second medical professional, an input including a treatment attribute corresponding to the radiation therapy treatment of the patient; generate a first radiotherapy proficiency score for the first medical professional and a second radiotherapy proficiency score for the second medical professional, the first score and the second score corresponding to a professional attribute of the first medical professional and the second medical professional; execute the machine-learning language processing model using the input to predict a contextual suggestion corresponding to the treatment attribute in accordance with the first radiotherapy proficiency score and the second radiotherapy proficiency score, wherein the machine-learning language processing model is trained using a set of transcriptions of a set of RTTP discussions for a set of previously implemented radiation therapy treatments; present, on the interaction graphical component, the contextual suggestion corresponding to the treatment attribute; and in response to receiving an indication of approval from the first medical professional and the second medical professional, transmit the treatment attribute to a radiation therapy plan optimizer.

In some aspects, the techniques described herein relate to a system, wherein the first radiotherapy proficiency score or the second radiotherapy proficiency score corresponds to a time period of medical experience of the first medical professional or the second medical professional.

In some aspects, the techniques described herein relate to a system, wherein the first radiotherapy proficiency score or the second radiotherapy proficiency score is generated using the machine-learning language processing model via analyzing a previous conversation associated with the first medical professional or the second medical professional.

In some aspects, the techniques described herein relate to a system, wherein the server is further configured to present, on the interaction graphical component, a hyperlink configured to direct the interaction graphical component to third-party data associated with the radiation therapy treatment.

In some aspects, the techniques described herein relate to a system, wherein the machine-learning language processing model is further trained using previously performed radiation therapy treatments.

In some aspects, the techniques described herein relate to a system, wherein the server is further configured to, in response to the input satisfying a threshold, present in the interaction graphical component, a warning message.

The server may be further configured to, in response to the second input satisfying a threshold, presenting, by the processor in the interaction graphical component, a warning message.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates the components of a system for conducting RTTP discussions and developing RTTPs using machine-learning language model processing, in accordance with an embodiment.

FIG. 2 shows an operational workflow of a method performed in hardware and software computing components that host and execute a machine-learning language processing in accordance with an embodiment.

FIG. 3 depicts an example user interface of collaboration software for an RTTP discussion to collaborate and interact with machine-learning language processing models, in accordance with an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting of the subject matter presented.

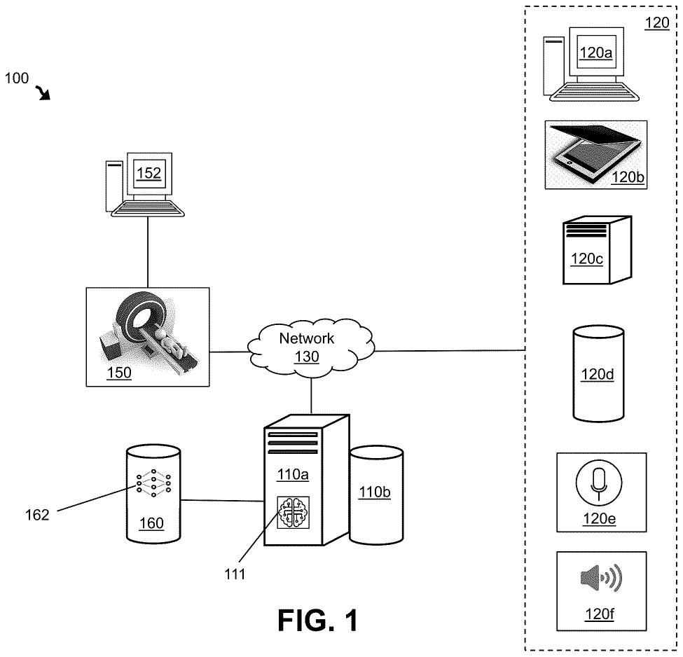

FIG. 1 illustrates components of a system 100 for conducting RTTP discussion and developing RTTPs using machine-learning language model processing, according to an embodiment. The system 100 may include an analytics server 110a, a system database 110b, a machine-learning language processing model 111 for presenting contextual information to participants of an RTTP discussion, end-user devices 120a-120f (collectively end-user devices 120), a medical device 150, a medical device computer 152, a database 160, and a radiotherapy plan optimizer 162. Various components depicted in FIG. 1 may belong to a radiation therapy treatment clinic at which patients may receive radiation therapy treatment, in some cases via one or more radiation therapy machines (e.g., the medical device 150).

The system 100 is not confined to the components described herein and may include additional or other components, not shown for brevity, which are to be considered within the scope of the embodiments described herein.

The above-mentioned components may be connected to each other through one or more networks 130. Examples of the network 130 may include, but are not limited to, private or public local-area networks (LAN), wireless local-area networks (WLAN), metropolitan-area networks (MAN), wide-area networks (WAN), and the Internet. The network 130 may include wired and/or wireless communications according to one or more standards and/or via one or more transport mediums. The communication over the network 130 may be performed in accordance with various communication protocols such as Transmission Control Protocol and Internet Protocol (TCP/IP), User Datagram Protocol (UDP), and IEEE communication protocols. In one example, the network 130 may include wireless communications according to Bluetooth specification sets or another standard or proprietary wireless communication protocol. In another example, the network 130 may also include communications over a cellular network, including, e.g., a GSM (Global System for Mobile Communications), CDMA (Code Division Multiple Access), or EDGE (Enhanced Data for Global Evolution) network.

The analytics server 110a may generate and display an electronic platform configured to interface a user with the machine-learning language processing model 111 and for receiving patient information and outputting the results of execution of the machine-learning language processing model 111 and the radiotherapy plan optimizer 162. The electronic platform may include graphical user interfaces (GUI) displayed on each of the end-user devices 120, the medical device 150, and/or the medical device computer 152. An example of the electronic platform generated and hosted by the analytics server 110a may be a web-based application or a website configured to be displayed on different electronic devices, such as mobile devices, tablets, personal computers, and the like.

The platform hosted on the analytics server 110a or other device of the system 100 includes collaboration software accessible to the user devices 120 of the participating members of the RTTP discussion. The collaboration software may include any type of software facilitating user-group collaborations, which may include live interaction software (e.g., teleconferencing software) or asynchronous collaborations (e.g., online postings). Non-limiting examples of the collaboration software may include MS Teams®, Skype®, WebEx®, Slack®, and Twilio®, among others. The analytics server 110a may execute an RTTP Application (RA) that comprises, invokes, executes, and manages the operations of the AI agent and machine-learning language processing model 111, among other functions. The RA collects and ingests various types of inputs and feeds the inputs into the AI agent and machine-learning language processing model 111.

In some embodiments, the RA (including the AI agent and machine-learning language processing model 111) is a software module component (e.g., plug-in) of the collaboration software of the platform of the analytics server 110a. In some embodiments, the collaboration software makes calls to the RA software of the analytics server 110a to provide inputs to and invoke operations of the AI agent and machine-learning language processing model 111.

The information displayed by the RA of the electronic platform can include, for example, input elements to receive data associated with a patient to be treated (e.g., plan objectives) and display results of predictions for AI-generated text for continuing the RTTP discussion, as produced by the machine-learning language processing model 111, which may include various formats of responsive predicted outputs (e.g., text, images, or videos generated in response to inputs received through the RA or electronic platform). Optionally, the outputs produced by the machine-learning language processing model 111 of the RA may be fed to the radiotherapy plan optimizer 162 (e.g., a predicted radiotherapy plan). The analytics server 110a may then display the results for the participants of the RTTP discussion at a user device 120 and/or another medical professional at the user device 150. In some embodiments, the medical device 150 can be a diagnostic imaging device or a treatment delivery device.

The analytics server 110a may be any computing device comprising a processor and non-transitory machine-readable storage capable of executing the various tasks and processes described herein. The analytics server 110a may employ various processors such as central processing units (CPU) and graphics processing unit (GPU), among others. Non-limiting examples of such computing devices may include workstation computers, laptop computers, server computers, and the like. While the system 100 includes a single analytics server 110a, the analytics server 110a may include any number of computing devices operating in a distributed computing environment, such as a cloud environment.

End-user devices 120 of an RTTP discussion or physicians may be any computing device comprising a processor and a non-transitory machine-readable storage medium capable of performing the various tasks and processes described herein. Non-limiting examples of an end-user device 120 may be a workstation computer, laptop computer, tablet computer, and server computer. In operation, various users may use end-user devices 120 to access the GUI operationally managed by the analytics server 110a. Specifically, the end-user devices 120 may include a clinic computer 120a, a clinic server 120b, and medical professional devices 120c, which may include any electronic devices operated by members of the RTTP discussion, medical professionals, and scientists that access and review various types of patient-related treatment data and RTTPs for the patient, among other types of data and information exchanges.

For instance, members of the RTTP discussion may operate the medical professional devices 120c to review patient-related treatment data to develop a consensus of a diagnosis and/or RTTP for the patient. Even though referred to herein as “end-user” devices, these devices may not always be operated by end-users. For instance, the clinic server 120b may not be directly used by an end-user. However, the results stored on the clinic server 120b may be used to populate various GUIs accessed by an end-user via the medical professional device 120c. Patient-related information generated by the various types of devices of the system 100, outside the context of RTTP discussion, may be stored in the system database 100b. The stored patient data may be referenced by the RA during RTTP discussion discussions and/or referenced by the RA for training the machine-learning language processing model 111 or the AI agents.

The medical device 150 may be a radiation therapy machine configured to implement a patient's radiotherapy treatment. The medical device 150 may also be in communication with a medical device computer 152 that is configured to display various GUIs discussed herein. For instance, the analytics server 110a may display the results predicted by the radiotherapy plan optimizer 162 onto the computing devices described herein.

The machine-learning language processing model 111 may be stored in the system database 110b. The machine-learning language processing model 111 may be configured or trained to automatically generate text, image, or video responses based on inputs received at a user interface or other types of inputs (e.g., speech captured at a conference room microphone or microphone of an end-user device 120). The machine-learning language processing model 111 can be configured and trained to receive the various inputs from the members of the RTTP discussion and patient attributes for a patient as input and automatically generate various predicted responsive outputs for continuing the discussion.

The user-provided inputs include, for example, text inputs entered by members of the RTTP discussion via user interfaces or audio signals containing speech audio of the members of the RTTP discussion captured by microphones of the user devices 120 or conference room. The RA may include Automated Speech Recognition (ASR) software that converts speech audio to written text. The ASR software comprises a machine-learning architecture trained to detect portions or frames of the audio signal containing the speech audio of a member speaker. The ASR also comprises and applies Natural Language Processing (NLP) layers of the machine-learning architecture that generates text-based output from the portions of the audio signal containing the detected speech audio. The text generated by the ASR may be fed as an input to the machine-learning language processing model 111. The machine-learning language processing model 111 is trained to simulate and contextually continue a conversation with the RTTP discussion, based upon the text of the RTTP discussion and the patient-related data indicating the patient attributes over time.

In some embodiments, the analytics server 110a may execute a radiotherapy plan optimizer 162 to generate one or more treatment attributes for an RTTP complying with any radiation therapy plan objectives based on patient attributes of a patient for which the radiotherapy treatment plan is being generated. The radiotherapy plan optimizer 162 can be stored in the database 160. The radiotherapy plan optimizer 162 can generate one or more treatment attributes, for example, by iteratively calculating one or more treatment attributes where, with each iteration, the radiotherapy plan optimizer 162 can revise the one or more treatment attributes of the RTTP in accordance with a cost value. The analytics server 110a may deploy the radiotherapy plan optimizer 162 to generate an RTTP for a patient based on patient attributes for the patient.

The radiotherapy plan optimizer 162 may iteratively calculate one or more treatment attributes of the RTTP. For instance, with each iteration, the radiotherapy plan optimizer 162 may generate a candidate RTTP having various attributes. The plan optimizer 162 may then use one or more loss functions to calculate a cost value for the generated candidate RTTP. The cost value may indicate a likelihood of the candidate RTTP violating a set of rules, whether internal and/or external rules. For instance, the cost value may indicate whether the candidate RTTP violates any of the plan objectives. The radiotherapy plan optimizer 162 may analyze the cost value. If needed (e.g., when the cost value satisfies a threshold), the radiotherapy plan optimizer 162 may revise the candidate RTTP and re-execute its loss function to generate a new cost value.

Depending on whether the new cost function is increasing or decreasing, the plan optimizer computer model may revise the candidate RTTP again and recalculate the cost value. The radiotherapy plan optimizer 162 may continue this iterative approach until converging upon an RTTP (or the final RTTP) that has a cost value that satisfies a threshold. In some implementations, the treatment attribute for the patient may also indicate how the radiotherapy treatment may be combined or sequentially implemented with other types of treatment modalities (e.g., surgery, chemotherapy).

The RTTP discussion may implement the features and functions of the analytics server 110a described herein for discussing potential, ongoing, or prior radiotherapy-based treatments, but embodiments are not so limited to discussions for potential radiotherapy treatments. For instance, the RTTP discussion may implement and benefit from the LLM and AI-related features of the system 100 when the RTTP discussion is discussing any number of potential treatment options (e.g., hot spots) and then ultimately decides that another treatment attribute should be used to generate the patient's RTTP. In some circumstances, for example, the analytics server 110a or other features of the system 100 may generate various outputs about the treatment options discussed by the RTTP discussion that ultimately dissuade the RTTP discussion from pursuing radiotherapy.

The system database 110b may contain data needed to train the machine-learning language processing model 111. For instance, the system database 110b may include data associated with previously treated patients, such as patient diagnosis data (e.g., tumor data or tumor location), biometric data (e.g., BMI, body weight, height, or various other bodily measurements), and the like. Additionally, the system database 110b may include RTTP discussion discussions (audio, transcription, and/or video recording of RTTP discussion meetings) corresponding to the previously treated patients as well. Therefore, the system database 110b may include all data associated with how the previously treated patients were diagnosed and treated. As described herein, the analytics server 110a may use the data stored within the system database 110b to train the machine-learning language processing model 111.

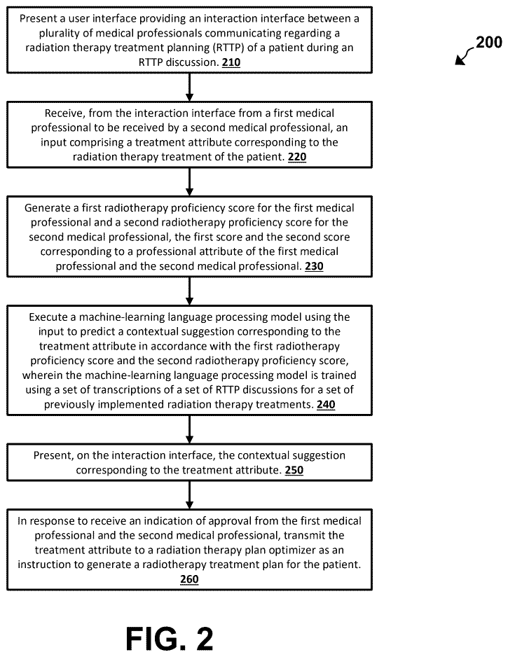

FIG. 2 shows an operational workflow of a method 200 performed in hardware and software computing components that host and execute a machine-learning language processing, in accordance with an embodiment. The method 200 may include steps 210-260. However, other embodiments may include additional or alternative steps or may omit one or more steps altogether. The method 200 is described as being executed by a server, such as the analytics server described in FIG. 1. However, one or more steps of the method 200 may be executed by any number of computing devices operating in the distributed computing system described in FIG. 1. For instance, one or more computing devices may locally perform some or all of the steps described in FIG. 2.

Using the method 200, the analytics server can implement a combination of a machine-learning language processing model of an RA for predicting responsive text-based outputs or other types of responsive outputs that contribute to RTTP discussion focused on developing an RTTP. To do so, the RA can host or provide an interaction graphical component (e.g., conference audiovisual inputs and outputs; a text-based collaboration interface) for capturing inputs from a conference room microphone or a computing device being accessed by a user (e.g., member-user of the RTTP discussion). In some cases, the analytics server hosting the RA retrieves patient-related data from a patient database. Through the interaction graphical component or by capturing various types of user inputs (e.g., microphone capturing voice speech of RTTP discussion members speaking during RTTP discussion meetings), the users can input patient attributes of a patient or any other data or the RA may query the patient-related data from the patient database.

The analytics server can receive the inputs and provide the inputs to the machine-learning language processing model. The machine-learning language processing model of the RA can process the inputs and generate responses (e.g., text, images, video, etc.) that continue and enrich the RA discussions based on the inputs provided by the members of the RTTP discussion or retrieved from the patient database.

At step 210, the analytics server may present a user interface providing an interaction graphical component between a plurality of medical professionals communicating regarding a radiation therapy treatment planning (RTTP) of a patient during an RTTP discussion.

The analytics server may present a user interface on a computing device. The analytics server may generate the user interface of the RA (or collaboration software having the RA) and transmit the user interface to the computing device over a network. The analytics server may generate and transmit the user interface to the computing device when providing an electronic platform that facilitates RTTP discussion regarding treatment considerations and planning (e.g., RTTP) for patients based on inputs from the members of the RTTP discussion.

The analytics server can generate the user interface to have an interaction graphical component. The interaction graphical component can be an interface that enables communication between a user viewing and providing inputs into an interaction graphical component and the analytics server or a machine-learning language processing model. The machine-learning language processing model can be a large language model that has been trained to generate text, image, and/or video responses to text, image, and/or video input. The machine-learning language processing model can be or include a neural network, such as a neural network with a transformer architecture. The interaction graphical component can enable a form of communication similar to a conversation between two humans that can involve back-and-forth exchanges of messages between participants in the RTTP discussion. In this case, the participants may be expert users who are members of the RTTP discussion, such as an oncologist or other clinician, and an AI agent that implements the machine-learning language processing model to ingest inputs from the discussion and provide the outputs of the machine-learning language processing model to the RTTP discussion interface.

In some implementations, the interaction graphical component can simulate an instant messaging application conversation between the experts and the machine-learning language processing model. For example, a user can input text into the interaction graphical component. The interaction graphical component can be a feed (e.g., a user feed). The text can be or include patient attributes for a patient receiving radiotherapy treatment. For example, the user can select a send button to cause the text to be transmitted to the analytics server. The analytics server can input the text, including the patient attributes, into the machine-learning language processing model and execute the machine-learning language processing model. Based on the input, the language machine-learning model may output a response in text or additional types of information, such as media image data containing image scans, charts/graphs, and the like.

The analytics server may present the response in the interaction graphical component. For instance, the response text may be presented in the interaction graphical component below the most recent input text by the user, thereby displaying the inputs and responses as a running “chat” interaction within the interaction graphical component. The user can respond to the response from the machine-learning language processing model with text following the response and submit the user input response. This process can repeat any number of times to simulate an instant message application conversation.

The interaction graphical component can cover a portion of the user interface of the collaboration software or RA or be a widget of the user interface of the collaboration software or RA. The user interface can include other portions or widgets that offer differing functionality, such as external software tools. While the interaction graphical component can enable communication between the RTTP discussion participants and the AI agent(s) implementing corresponding machine-learning language processing model(s), the analytics server can also include a portion of the user interface that displays, for example, different patient attributes of patients, treatment options mentioned in the RTTP discussion, or other types of outputs generated by a machine-learning language processing model. For example, the RA may capture an audio signal in which a participating member mentions a particular patient attribute, treatment option, or other aspect of the patient's treatment planning and convert the audio signal into a text input for the AI agent.

The machine-learning language processing model and the AI agent may be trained to, for example, provide additional information about the particular patient attribute, treatment option, or other aspect of the patient's treatment planning mentioned by the participating member. The AI agent may include this additional information as an output to the user interface presenting the ongoing RTTP discussion.

The RA may receive an input indicating an identifier (e.g., a name) of the patient into the user interface or the platform provided by the analytics server. Responsive to receiving the input, the analytics server can retrieve patient data (e.g., one or more patient attributes) regarding the patient from non-transitory memory containing database records of the patient database. Examples of patient attributes the analytics server may retrieve include computed tomography (CT) scans of the patient or a tumor of the patient, images of the patient or a tumor of the patient, previously collected patient attributes of the patient, such as data collected from previous health tests, among others.

The analytics server can present the retrieved patient data on the user interface in another widget or a portion of the user interface separate or adjacent to the interaction graphical component. In some cases, the portion or widget of the user interface, including the retrieved patient data, can be separated from the interaction graphical component on the user interface by a line (e.g., a vertical line going across the width or length of the user interface (e.g., along the x-axis or the y-axis) of the user interface).

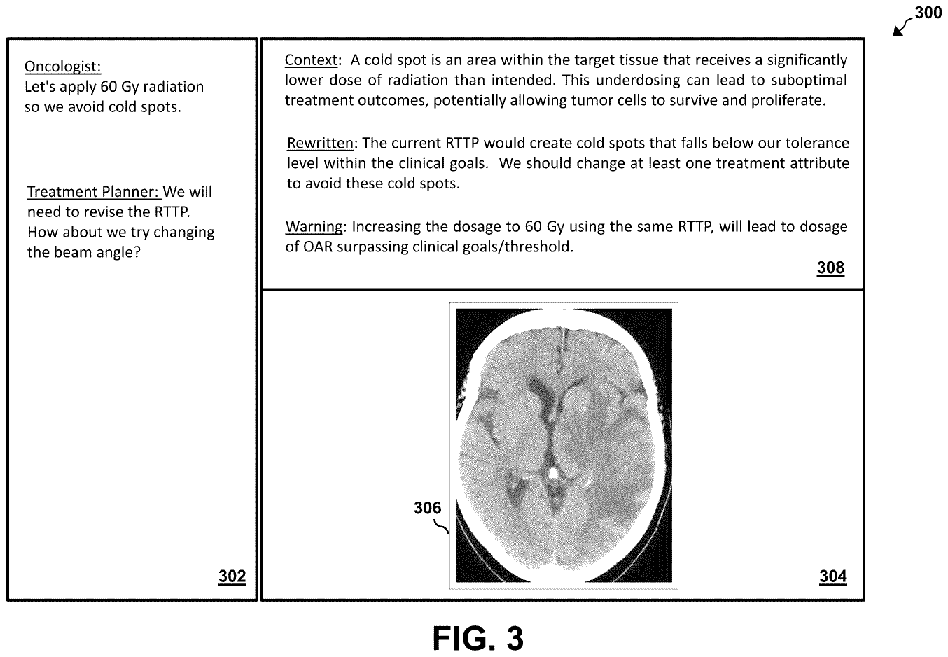

In a non-limiting example, referring now to FIG. 3, the analytics server can generate a user interface 300 to include an interaction graphical component 302 and a patient data interface 304 of a platform hosting collaboration software and an RA for contributing to RTTP discussion discussions. The interaction graphical component 302 can be a portion of the user interface 300 that users (e.g., members of an RTTP discussion participating in the discussion) can operate or access to interact with a machine-learning language processing model. The user or the RA can input text, images, and/or video into the interaction graphical component 302 and receive responses from the machine-learning language processing model in the interaction graphical component 302.

The patient data interface 304 can be an area of the user interface 300 through which the analytics server (e.g., AI agent and machine-learning language processing model) can present retrieved patient data 306 for a patient (e.g., CT image of the PTV). The patient data interface 304 can be configured to present any type of data, such as text, images, or videos, which may include various types of data relevant to the RTTP discussion discussions. The analytics server can generate the user interface 300 and transmit the user interface to a computing device accessed by a user. In some cases, the auxiliary interface 308 may present a data feed containing AI-originated text or data. Additionally or alternatively, the interaction graphical component 302 may present the data feed having text or other data of corresponding human response or request inputs.

The user interface can include an auxiliary interface 308 to illustrate additional information relevant to the conversation on the interaction graphical component 302 and/or for the retrieved data on the patient data interface 304 to the RTTP discussion members using the user interface 300. The auxiliary interface 308 can include contextual data generated by the analytics server and discussed herein. Moreover, the auxiliary interface 308 may include warning messages.

Referring back to FIG. 2, at step 220, the analytics server may receive, from the interaction graphical component from a first medical professional to be received by a second medical professional, an input comprising a treatment attribute corresponding to the radiation therapy treatment of the patient.

The analytics server may receive, from the interaction graphical component, a first input comprising a patient attribute of the patient and/or another input corresponding to the radiation therapy treatment of the patient. The analytics server can receive the first input from the interaction graphical component of the user interface. The first input can include a first patient attribute of a patient. The first input can include the patient attribute and any number of other patient attributes. The first patient attribute and the other patient attributes can be any type of patient attribute, such as, for example, height, gender, weight, treatment options for the patient, treatment attributes (e.g., gantry movements, gantry positions, etc.), treatment objectives, attributes regarding a tumor (e.g., size or shape), images of the patient or tumor, tumor stage, the primary site of treatment, endpoints, whether the tumor has been extended, body mass index, blood pressure, medical history (e.g., previous medical treatments received by the patient, etc.). A user can provide the first input and select a send or submit button on the user interface, for example. The analytics server can receive the first input from the computing device presenting the user interface.

In one example, the user can provide the first input by typing the first patient attribute and any other patient attributes of the first input into the interaction graphical component. For example, the user can type the current height, age, weight, and blood pressure into the interaction graphical component. Typing the patient attributes into the interaction graphical component can cause text to appear on the interaction graphical component. The user can select a submit button responsive to typing the first input into the interaction graphical component to submit the first input to the analytics server.

In another example, the user can provide the first input by selecting the first attribute and any other patient attributes from patient data that is presented on the user interface. For example, the user can select an image of a tumor of the patient and/or one or more other patient attributes from the patient data. The user can select an option to move the selected patient attributes into the interaction graphical component or otherwise drag the selected patient attributes into the interaction graphical component. Upon moving the selected patient attributes into the interaction graphical component, the user can select an option to submit or send the selected patient attributes to the analytics server. In some cases, the user can select an option to submit or send the selected patient attributes without moving the selected patient attributes to the interaction graphical component.

In some cases, the user can provide the first input by typing the one or more patient attributes into the interaction graphical component and selecting one or more patient attributes from the patient data for the patient displayed on the user interface. The user can type one or more patient attributes into the interaction graphical component, and select one or more patient attributes from the patient data, and select a submit button to transmit the first input to the analytics server.

Using this input, the analytics server may retrieve patient data, such that the analytics server, via the machine-learning language processing model, can determine contextual data/suggestions regarding the patient.

The analytics server may also receive a treatment attribute from one medical professional participating in the RTTP discussion. As used herein, a treatment attribute is any attribute that can be used to generate the RTTP for the patient.

Non-limiting examples of a treatment attribute may include patient information (e.g., patient demographics, such as age, gender, medical history, previous treatments and outcomes, current health status and any comorbidities, tumor characteristics, tumor type and histology, tumor size, shape, and location, tumor stage, and grade, presence of metastases), imaging data (e.g., CT, MRI, and PET scans, tumor and organ delineations, spatial relationships between tumor and surrounding tissues), dose distribution (e.g., prescribed dose to the tumor (target volume), dose constraints for surrounding critical organs and tissues, dose-volume histograms (DVHs)), field geometry (e.g., beam angles and orientations, field sizes and shapes, MLC (multi-leaf collimator) settings), optimization parameters (e.g., treatment modalities, such as IMRT, VMAT, proton therapy, optimization objectives, and constraints, weighting factors for different treatment goals), technical specifications (e.g., equipment capabilities and limitations, treatment machine calibration and settings, software version and capabilities), and/or clinical protocols (e.g., standard treatment protocols and guidelines, protocol-specific dose constraints and objectives, and the like.

The treatment attributes may collectively contribute to developing a comprehensive and effective radiotherapy treatment plan tailored to the individual patient's needs and specific clinical scenarios.

Referring again to FIG. 3, in a non-limiting example, the user can provide the first input into the interaction graphical component 302. The user can do so, for example, by typing the first patient attribute and any number of other patient attributes into the interaction graphical component 302. In some cases, in addition to or instead of typing the first patient attribute into the interaction graphical component 302, the user can select the first patient attribute and/or any number of other patient attributes from the patient data interface 304. The user can select a submit button on the user interface 300 or otherwise submit the patient attributes by selecting an input/output button to transmit the first patient attribute and any other patient attributes to the analytics server. Additionally, the medical professionals can provide a treatment attribute for the patient. As depicted, the oncologist may indicate that the dosage may need to be increased to 60 Gy. Therefore, in this example, the 60 Gy is a treatment attribute.

Referring back to FIG. 2, at step 230, the analytics server may generate a first radiotherapy proficiency score for the first medical professional and a second radiotherapy proficiency score for the second medical professional, the first score and the second score corresponding to a professional attribute of the first medical professional and the second medical professional.

The analytics server may generate a first radiotherapy proficiency score for the first medical professional and a second radiotherapy proficiency score for the second medical professional. These proficiency scores may be determined based on various professional attributes associated with each medical professional participating in the RTTP discussion. The professional attributes may include factors such as the individual's years of experience, specific training, past performance metrics, familiarity with particular treatment protocols, and overall expertise in the field of radiotherapy.

The radiotherapy proficiency score may be a numerical value generated by the analytics server to represent the level of expertise and capability of a medical professional in handling radiation therapy treatment planning tasks. This score is calculated based on various factors, such as the individual's years of experience, specific training and certifications, past performance metrics, and familiarity with particular treatment protocols. Essentially, it quantifies the professional attributes of each medical professional, providing a standardized measure of their proficiency.

As discussed herein, the radiotherapy proficiency score may be used by the analytics server (or the machine-learning language processing model) to tailor communications. For example, when an oncologist sends a treatment plan request, the radiotherapy proficiency score of the recipient (such as a physicist) is referenced to adjust the message's terminology, level of detail and specificity. A higher proficiency score would prompt the system to generate a more concise message, assuming the recipient's familiarity with standard protocols, while a lower score would result in more detailed and explanatory communication. This approach ensures that the communication is clear, appropriate, and effective, based on the recipient's expertise level.

The radiotherapy proficiency level of the medical professionals may be identified using a combination of factors that are analyzed by the analytics server. These factors may include years of experience, specific training and certifications, past performance metrics, and familiarity with particular treatment protocols. The analytics server may aggregate this data to generate a proficiency score for each medical professional. For instance, an oncologist with extensive experience and specialized training in advanced radiation therapy techniques would receive a higher proficiency score than a newly certified oncologist. Similarly, a physicist with a long track record of handling complex treatment plans would be scored higher than a recent graduate.

In some embodiments, the proficiency score may correspond to a vector (e.g., a multi-dimensional vector) that can represent different values for different attributes of a medical professional discussed herein. For instance, instead of representing a medical professional as a single scalar value, the medical professional may be evaluated for a defined number of attributes where each evaluation in each attribute may be used to generate a vector corresponding to the medical professional's score. Therefore, the score, as used herein, does not need to be limited to one attribute of the medical professional and can represent multiple attributes at the same time. In this way, different aspects of a medical professional may be represented, such that the medical professional can be evaluated (in totality) with others involved in the treatment of the patient.

By assessing and quantifying these attributes, the analytics server can assign a proficiency score that reflects the competency and capability of each professional in handling radiotherapy treatment planning tasks. This scoring mechanism may ensure that the communication and instructions provided are tailored to the expertise level of the recipient, thereby enhancing the clarity and effectiveness of the information exchanged between the professionals.

In some embodiments, the analytics server may retrieve a profile associated with the medical professionals participating during the RTTP discussion. The analytics server may then generate the radiotherapy proficiency using various scoring schemes.

In some embodiments, the analytics server may use the machine-learning model (sometimes the same model as the language model discussed herein) to predict a radiotherapy proficiency score for the medical professionals. For instance, the machine-learning model may ingest the RTTP transcript (for example the terminology used in discussions, questions asked in discussions, answers given to questions) and predict a score for the participants. In those embodiments, the machine-learning model may be trained by ingesting previously conducted RTTP discussions and previously implemented treatments (corresponding to the same RTTP discussions). Accordingly, by ingesting how a medical professional discusses treating a patient, the machine-learning model may predict their radiotherapy proficiency score.

At step 240, the analytics server may execute a machine-learning language processing model using the input to predict a contextual suggestion corresponding to the treatment attribute in accordance with the first radiotherapy proficiency score and the second radiotherapy proficiency, wherein the machine-learning language processing model is trained using a set of transcriptions of a set of RTTP discussions for a set of previously implemented radiation therapy treatments.

The analytics server may leverage a pre-trained and/or specialized machine-learning language processing model to convert RTTP communications into more precise and contextually appropriate formats. When a medical professional, such as an oncologist, inputs a message into the system, the machine-learning language processing model first assesses the recipient's expertise and experience level (by calculating the proficiency score), whether they are a physicist, dosimetrist, or another specialist. Using a specially designed prompt, the machine-learning language processing model may generate contextual data associated with the input that is appropriate in light of the proficiency score of the recipient. For instance, the machine-learning language processing model may rewrite the input message to include more exact terminology and necessary details tailored to the recipient's needs. This ensures that the communication is clear and unambiguous, addressing common issues of misinterpretation that can arise from varied professional backgrounds and terminologies.

The analytics server may execute the machine-learning language processing model using the input of the treatment attribute and/or the entire communication received from the first medical professional. The machine-learning language processing model may then provide contextual data associated with the RTTP and/or the treatment attribute. In some embodiments, the contextual data may include an augmented version of the same communication provided by the first medical professional. Additionally, or alternatively, the contextual data may include a definition of the communication received by the first medical professional. In some embodiments, the contextual data may include the same input provided by the first medical professional rewritten in a form that is more suitable in accordance with the proficiency score of the second medical professional.

The machine-learning language processing model may be trained on a set of transcriptions (sometimes referred to as “discussion logs”) of a set of RTTP discussion review discussions stored in one or more databases for a set of previously implemented RTTPs for other patients. In those embodiments, the RTTP discussion and a predicted (or known) proficiency score of each participant may be used to train the model.

The machine-learning language processing model can use one or more templates to determine the contextual data in view of the proficiency score of each participant in the RTTP discussion. In some cases, one or more of the templates can include a list of types of professional attributes or proficiency scores. For instance, different templates may be specific to medical professionals within a defined range of proficiency score ranges (e.g., more experienced medical professionals). In a non-limiting example, a template can correspond to how highly proficient medical professionals communicate.

The analytics server can generate or train the machine-learning language processing model using supervised learning, unsupervised learning, or semi-supervised learning techniques. For example, the analytics server can train the machine-learning language processing model using a labeled training data set. The labeled training data set may include different sentences or paragraphs of text that correspond to the radiotherapy treatment of patients. The sentences or paragraphs of text may correspond to the radiotherapy treatment of patients, for example, because the sentences or paragraphs can include values of one or more patient attributes and may include specific keywords that correspond to radiotherapy treatment (e.g., radiotherapy, radiation, radiation therapy, oncology, tumor, radiation oncology, linear accelerator, radiation dose, external beam radiation), among others.

In some cases, the labeled training data set can correspond to radiotherapy based on the source of the training data. For example, the labeled training data set can include sentences or text from clinical guidelines for radiotherapy treatment planning, case studies of radiotherapy treatment, medical journals and research papers on radiation therapy treatment, structured radiotherapy treatment plans, etc. The labeled training data set can include annotations indicating the rationale behind certain decisions in the treatment plans or any specific considerations that were taken into account. The labeled training data set can additionally or instead include labels indicating the correct and unambiguous terms referring to otherwise ambiguous terms. The analytics server can automatically label the training data set, or a human reviewer can label the training data set. Such text can be fed (e.g., by the analytics server) into the machine-learning language processing model for training.

During training, the analytics server may iteratively execute the machine-learning language processing model to generate new predicted text, images, and/or videos based on the training dataset (e.g., for each entry of text, images, and/or videos). If the predicted results do not match the desired outcome, the analytics server can continue the training unless and until the computer-generated recommendation satisfies one or more accuracy thresholds and is within an acceptable range.

Using the method 200, the machine-learning language processing model discussed herein may refine user communication within the RTTP discussion by making it more specific or adjusting its conciseness. In practice, this means any user-provided communication, whether directed to another human expert or a software component (such as another LLM-powered interface), is enhanced using a specialized prompt. Additionally, the converted communication will be displayed to the user, allowing them to review its accuracy and request corrections if necessary. For instance, in some embodiments, the contextual data provided may be a more concise or exact version of the same instructions provided by the medical professional.

In some embodiments, the analytics server may generate a prompt and then transmit the prompt (along with the input from the medical professional) to the machine-learning language processing model. For instance, the prompt may be “The following text is a part of RT oncologist communication for a physicist. Please rewrite the text using more exact language and more specific terms: {user input}.” In this way, the prompt will provide a text that should have the same meaning as the original user communication but using more exact terminology.

In some embodiments, the prompt may address the level of conciseness of the RTTP input by the medical professional. For instance, the analytics server may generate the following prompt:

“The following communication by a physician requires more details for a physicist: {user input}” or “The following communication of the physicist needs to be changed to a more concise format: {user input}.”

In some embodiments, the prompts may include more radiotherapy-specific detail and can also contain information related to the particular individuals (like examples of the desired level of descriptions/conciseness identified or predicted based on the proficiency score).

Additionally, or alternatively, the machine-learning language processing model may be trained to identify the terms in the input (e.g., discussion in the RTTP discussion) that can be misinterpreted. For instance, using the proficiency score of the recipient, the machine-learning language processing model may determine that a certain term might be ambiguous to the recipient. As a result, the machine-learning language processing model may replace the term that is predicted to be ambiguous to the recipient with a more precise term. In some embodiments, the machine-learning model may also provide contextual data regarding the potentially ambiguous term. In a non-limiting example, an oncologist may input, “We need to change a couple of attributes like the angle because the PTV is going to have too much radiation.” In this example, the machine-learning language processing model may determine that “too much radiation” is referring to a “hot spot.” As a result, the contextual data provided in the RTTP discussion may be the same sentence rewritten in a more conscience form, such as “we must change one or more treatment attributes, such as a beam angle because the planning target volume (e.g., tumor) might have a hot spot that exceeds one or more clinical goals.” As depicted in FIG. 3, the provided contextual data included more concise language and provided at least one alternative to the original communication in the RTTP discussion. For instance, the same text provided by the oncologist can be rewritten by the analytics server.

In some embodiments, the revised text may be presented to the sender instead of the original text. For instance, when a first medical professional inputs a text, the analytics server may provide the revised text to the sender before transmitting the text to the intended recipient. Using various editing tools, the sender may revise the suggested text and/or approve the suggested text. At that time, the analytics server may transmit the text to the recipient. In this way, the text is already revised by the time the recipient is viewing the communication.

In some embodiments, the user interface presenting the transformed text or the contextual data can then use visual attributes to distinguish the new language or the terms that have been revised, such as by highlighting the terms or providing drop-down boxes to allow one to choose among the proposed alternatives. In a non-limiting example, when a sender types a message, the analytics server may display a drop-down menu next to a few highlighted terms. When the analytics server identifies that the sender has interacted with the drop-down menu, the analytics server may display a list of proposed alternative phrases (e.g., “hot spot” instead of “too much dose”). In this way, the sender may revise the communication before it is transmitted to the recipient.

In some embodiments, the model may be trained using a combination of general-purpose models and specialized datasets specific to radiotherapy treatment planning. Initially, a standard LLM may be employed, which has been pre-trained on a diverse range of texts to understand and generate human-like language. This foundational model may then be further trained using a specialized training set that includes examples of communication between medical professionals in the context of radiotherapy (e.g., RTTP discussions). These examples may encompass various scenarios detailing how oncologists, physicists, and dosimetrists interact, ensuring the model learns the precise terminology, common practices, and contextual nuances required for accurate and effective communication in this specialized field.

In some embodiments, the machine-learning language processing model may be trained using a training set that contains examples of specialized or preferred terms. The terms can be collected and aggregated into a dictionary that would facilitate the production of new versions of the machine-learning language processing model on a regular basis. In some embodiments, this specialized model may be created by starting from a general-purpose LLM and training it further (modifying the internal model parameters) using a specialized training set.

Additionally, or alternatively, the analytics server may monitor and collect different interaction data (e.g., user-give text and augmented contextual data) and re-train the model accordingly. To enhance the model's capability, the analytics server may continuously augment its training data by collecting pairs of texts: the original user inputs and the model-generated outputs after user review and corrections. This iterative process allows the model to learn from real-world use cases, adapting to individual users'specific communication styles and preferences over time. By incorporating these real-world interactions, the model becomes increasingly proficient at generating contextually appropriate and precise communication tailored to the expertise levels and specific needs of different medical professionals involved in radiotherapy treatment planning.

At step 250, the analytics server may present, on the interaction graphical component, the contextual suggestion corresponding to the treatment attribute. The analytics server may display the contextual data generated via the machine-learning language processing model. Referring again to FIG. 3, in a non-limiting example, the analytics server can present the contextual data on the user interface 300. Specifically, the auxiliary interface 308 can be populated with text and/or images that correspond to the contextual data identified by the machine-learning language processing model.

In some embodiments, the analytics server may also display a hyperlink where the medical professional can be directed to a third-party website (or other electronic resource) that provides additional data associated with the treatment attribute pertinent to the RTTP discussion.

Referring back to FIG. 2, at step 260, the analytics server may, in response to receiving an indication of approval from the first medical professional and the second medical professional, transmit the treatment attribute to a radiation therapy plan optimizer, e.g., as an instruction to generate a radiotherapy treatment plan for the patient. In response to receiving an indication of approval from one or more (e.g., all) of the participants of the RTTP discussion, the analytics server may transmit the treatment attribute to a radiation therapy plan optimizer as an instruction to generate a radiotherapy treatment plan for the patient.

The radiotherapy plan optimizer can be a computer model (e.g., an optimization computer model) that is configured to generate one or more treatment attributes for a radiotherapy treatment plan that complies with the radiation therapy plan objectives for a patient (e.g., objectives in patient attributes of the patient) based on patient attributes of the patient for which the radiotherapy treatment plan is being generated. The radiotherapy plan optimizer can generate one or more treatment attributes, for example, by iteratively calculating one or more treatment attributes where, with each iteration, the radiotherapy plan optimizer revises the one or more attributes of the radiotherapy treatment plan in accordance with a cost value.

In some embodiments, the analytics server can generate a vector of tokens from the patient attributes. The analytics server can generate the vector of tokens using a tokenization algorithm, such as WordPiece or Byte Pair Encoding. In some cases, the analytics server can use the same tokenization algorithm that was used to train the radiotherapy plan optimizer to generate treatment attributes of a radiotherapy treatment plan. In generating the tokens, the analytics server can generate an array or vector of numbers from the patient attributes. In some cases, each attribute may correspond to a different number (e.g., map to a different number), and the analytics server can determine a number or token for each attribute based on the mappings. The analytics server can generate the vector of tokens from the patient attributes and transmit the vector of tokens to the radiotherapy plan optimizer.

In some embodiments, the analytics server can convert the vector of tokens to a structured data set. The structured dataset may include the data in the vector of tokens in a format that the radiotherapy plan optimizer can process. For example, the analytics server can convert the vector of tokens to a traditional structured cost function or field geometry definitions. For example, the analytics server can store a mapping of tokens to values of a cost function or field geometry settings. The analytics server can convert the vector of tokens to the traditional structure cost function or field geometry definitions based on the mapping. The analytics server can transmit the converted vector of tokens to the radiotherapy plan optimizer. In another example, the vector of tokens can contain a reference to one or more pre-defined treatment protocols (e.g., treatment protocol templates). Such tokens can be used to construct instructions to the radiotherapy plan optimizer. For example, in some cases, the token may instruct the plan optimizer to exclude and/or include certain treatment attributes from the radiotherapy treatment plan.

The radiotherapy plan optimizer can receive the patient attributes (e.g., the raw patient attributes, the vector of tokens representing the patient attributes, or the converted vector of tokens, as described above) and the treatment attribute provided within the RTTP discussion. A computer (e.g., the analytics server or another computer) storing the radiotherapy plan optimizer can execute the radiotherapy plan optimizer using the patient attributes as input. The radiotherapy plan optimizer can output one or more treatment attributes of a radiotherapy treatment plan for the patient based on the patient's attributes. The radiotherapy plan optimizer can transmit the one or more treatment attributes to the analytics server.

The analytics server can receive the one or more treatment attributes provided within the RTTP discussion. The analytics server can present the radiotherapy treatment plan (e.g., the one or more treatment attributes of the radiotherapy treatment plan) on the user interface. The analytics server can present the radiotherapy treatment plan on the interaction graphical component of the user interface or on another portion of the user interface. In some embodiments, the analytics server can transmit the one or more treatment attributes to a radiotherapy treatment machine to use to provide treatment to the patient. The radiotherapy treatment machine can operate (e.g., automatically operate) based on the one or more treatment attributes.

In some cases, the user at the user interface may adjust the radiotherapy treatment plan. For example, the user viewing the radiotherapy treatment plan at the user interface can provide an input (e.g., a text input or a selection of a button) at the user interface that the radiotherapy treatment plan is incorrect. The analytics server can receive the input indicating the radiotherapy treatment plan is incorrect.

In some embodiments, the analytics server may present (within the RA) a warning message indicating that the treatment attribute provided by the medical professionals may violate one or more clinical goals. Referring to FIG. 3 again, the analytics server may communicate with the plan optimizer and instruct the plan optimizer to re-execute the RTTP using 60 Gy dosage. As a result, the plan optimizer may inform the analytics server that using 60 Gy may then lead to an OAR dosage that is higher than a defined threshold (e.g., clinical goal). As a result, the analytics server may output the warning depicted within the auxiliary interface 308.

In a non-limiting example, an oncologist wants to give a radiation treatment plan to a physicist. The oncologist wants to prescribe a 64 Gray treatment for a patient's prostate but isn't sure how detailed the instructions should be since different physicists have different levels of experience. The initial communication from the oncologist is straightforward: “Please create a 64 Gray prostate treatment plan.”

Using the machine-learning language processing model discussed herein, this initial message is transformed to fit the experience level of the recipient. For a junior physicist, the model might output: “Please create a 64 Gray prostate treatment plan. The dose should be delivered to a specific point in the prostate, with a possible variation of plus or minus 5%. This point is in the middle of the radiation target, following standard practice. Make sure to follow and record all safety steps.” The augmented version may be shared with the oncologist and may only be transmitted to the physicist upon the oncologist's approval. The augmented version includes more detailed instructions and safety protocols to ensure clarity.

For a senior physicist, the model would generate a more concise message: “Please create a 64 Gray prostate treatment plan, following standard protocols. The dose should be to the specific point.” This shorter version assumes the senior physicist's familiarity with standard procedures, reducing unnecessary details.

The process starts with the oncologist typing their initial message into an interface of an RTTP discussion. The model then determines the recipient's proficiency score and adjusts the message accordingly. The oncologist reviews the updated message and can approve it or request further changes. Once approved, the detailed message is sent to the physicist. This method ensures that the instructions are clear and appropriate for the recipient's experience level, minimizing misunderstandings and making the treatment planning process more efficient.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc., may be passed, forwarded, or transmitted via any suitable means, including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What we claim is:

1. A method comprising:
presenting, by a processor, a user interface providing an interaction graphical component between a plurality of medical professionals communicating regarding a radiation therapy treatment planning (RTTP) of a patient during an RTTP discussion;
receiving, by the processor from the interaction graphical component from a first medical professional to be received by a second medical professional, an input comprising a treatment attribute corresponding to the radiation therapy treatment of the patient;
generating, by the processor, a first radiotherapy proficiency score for the first medical professional and a second radiotherapy proficiency score for the second medical professional, the first score and the second score corresponding to a professional attribute of the first medical professional and the second medical professional;
executing, by the processor, a machine-learning language processing model using the input to predict a contextual suggestion corresponding to the treatment attribute in accordance with the first radiotherapy proficiency score and the second radiotherapy proficiency, wherein the machine-learning language processing model is trained using a set of transcriptions of a set of RTTP discussions for a set of previously implemented radiation therapy treatments;
presenting, by the processor on the interaction graphical component, the contextual suggestion corresponding to the treatment attribute; and
in response to receiving an indication of approval from the first medical professional and the second medical professional, transmitting, by the processor, the treatment attribute to a radiation therapy plan optimizer.

2. The method of claim 1, wherein the first radiotherapy proficiency score or the second radiotherapy proficiency corresponds to a time period of medical experience of the first medical professional or the second medical professional.

3. The method of claim 1, wherein the first radiotherapy proficiency score or the second radiotherapy proficiency is generated using the machine-learning language processing model via analyzing a previous conversation associated with the first medical professional or the second medical professional.

4. The method of claim 1, further comprising presenting, by the processor on the interaction graphical component, a hyperlink configured to direct the interaction graphical component to third-party data associated with the radiation therapy treatment.

5. The method of claim 1, wherein the machine-learning language processing model is further trained using previously performed radiation therapy treatments.

6. The method of claim 1, further comprising in response to the input satisfying a threshold, presenting, by the processor in the interaction graphical component, a warning message.

7. The method of claim 1, wherein the first input is a medical image.

8. A system, comprising:
a server comprising at least one processor and a non-transitory computer-readable medium containing instructions that, when executed by the processor, causes the at least one processor to:
present a user interface providing an interaction graphical component between a plurality of medical professionals communicating regarding a radiation therapy treatment planning (RTTP) of a patient during an RTTP discussion;
receive, from the interaction graphical component from a first medical professional to be received by a second medical professional, an input comprising a treatment attribute corresponding to the radiation therapy treatment of the patient;
generate a first radiotherapy proficiency score for the first medical professional and a second radiotherapy proficiency score for the second medical professional, the first score and the second score corresponding to a professional attribute of the first medical professional and the second medical professional;
execute a machine-learning language processing model using the input to predict a contextual suggestion corresponding to the treatment attribute in accordance with the first radiotherapy proficiency score and the second radiotherapy proficiency, wherein the machine-learning language processing model is trained using a set of transcriptions of a set of RTTP discussions for a set of previously implemented radiation therapy treatments;
present, on the interaction graphical component, the contextual suggestion corresponding to the treatment attribute; and
in response to receiving an indication of approval from the first medical professional and the second medical professional, transmit the treatment attribute to a radiation therapy plan optimizer.

9. The system of claim 8, wherein the first radiotherapy proficiency score or the second radiotherapy proficiency corresponds to a time period of medical experience of the first medical professional or the second medical professional.

10. The system of claim 8, wherein the first radiotherapy proficiency score or the second radiotherapy proficiency is generated using the machine-learning language processing model via analyzing a previous conversation associated with the first medical professional or the second medical professional.

11. The system of claim 8, wherein the instructions further cause the processor to present, on the interaction graphical component, a hyperlink configured to direct the interaction graphical component to third-party data associated with the radiation therapy treatment.

12. The system of claim 8, wherein the machine-learning language processing model is further trained using previously performed radiation therapy treatments.

13. The system of claim 8, wherein the instructions further cause the at least one processor to, in response to the input satisfying a threshold, present in the interaction graphical component, a warning message.

14. The system of claim 8, wherein the first input is a medical image.

15. A system, comprising:
a computer configured to display a user interface; and
a server in communication with the computer and a machine-learning language processing model, the server configured to:
present the user interface providing an interaction graphical component between a plurality of medical professionals communicating regarding a radiation therapy treatment planning (RTTP) of a patient during an RTTP discussion;
receive, from the interaction graphical component from a first medical professional to be received by a second medical professional, an input comprising a treatment attribute corresponding to the radiation therapy treatment of the patient;
generate a first radiotherapy proficiency score for the first medical professional and a second radiotherapy proficiency score for the second medical professional, the first score and the second score corresponding to a professional attribute of the first medical professional and the second medical professional;
execute the machine-learning language processing model using the input to predict a contextual suggestion corresponding to the treatment attribute in accordance with the first radiotherapy proficiency score and the second radiotherapy proficiency, wherein the machine-learning language processing model is trained using a set of transcriptions of a set of RTTP discussions for a set of previously implemented radiation therapy treatments;
present, on the interaction graphical component, the contextual suggestion corresponding to the treatment attribute; and
in response to receiving an indication of approval from the first medical professional and the second medical professional, transmit the treatment attribute to a radiation therapy plan optimizer.

16. The system of claim 15, wherein the first radiotherapy proficiency score or the second radiotherapy proficiency corresponds to a time period of medical experience of the first medical professional or the second medical professional.

17. The system of claim 15, wherein the first radiotherapy proficiency score or the second radiotherapy proficiency is generated using the machine-learning language processing model via analyzing a previous conversation associated with the first medical professional or the second medical professional.

18. The system of claim 15, wherein the server is further configured to present, on the interaction graphical component, a hyperlink configured to direct the interaction graphical component to third-party data associated with the radiation therapy treatment.

19. The system of claim 15, wherein the machine-learning language processing model is further trained using previously performed radiation therapy treatments.

20. The system of claim 15, wherein the server is further configured to, in response to the input satisfying a threshold, present in the interaction graphical component, a warning message.
